(19) United States
(12) Patent Application Publication
(10) Pub. No.: US 2023/0270987 A1
(43) Pub. Date: Aug. 31, 2023

(54) Vascular Access Instrument

(71) Applicant: Becton, Dickinson and Company, Franklin Lakes, NJ (US)

(72) Inventors: Curtis H. Blanchard, Herriman, UT (US); Weston F. Harding, Lehi, UT (US)

(21) Appl. No.: 18/114,352

(22) Filed: Feb. 27, 2023

Related U.S. Application Data
(60) Provisional application No. 63/314,777, filed on Feb. 28, 2022.

(51) Int. Cl. A61M 39/02

(52) U.S. Cl. CPC A61M 39/0247; A61M 2039/0258

(57) ABSTRACT

An instrument configured to be inserted through a vascular access device includes a helical coil formed by a wire, with the helical coil defining a central passage, and a core wire extending along a longitudinal axis of the helical coil through at least a portion of the central passage of the helical coil. At least a portion of the core wire includes a planar surface extending in a direction along the longitudinal axis and a semi-cylindrical surface extending in a direction along the longitudinal axis.

### CROSS-REFERENCE TO RELATED APPLICATION

The present application claims priority to U.S. Provisional Application Ser. No. 63/314,777, entitled “Vascular Access Instrument”, filed Feb. 28, 2022, the entire disclosure of which is hereby incorporated by reference in its entirety.

### BACKGROUND OF THE INVENTION

### Field of the Invention

The present disclosure relates to a vascular access system with a secondary catheter.

### Description of Related Art

Catheters are frequently utilized to administer fluids into and out of the body. Patients in a variety of settings, including in hospitals and in home care, receive fluids, pharmaceuticals, and blood products via a vascular access device inserted into a patient's vascular system. Catheters of various types and sizes have been used extensively in a variety of procedures including, but not limited to, treating an infection, providing anesthesia or analgesia, providing nutritional support, treating cancerous growths, maintaining blood pressure and heart rhythm, and many other clinical uses. A common vascular access device is a plastic catheter that is inserted into a patient's vein. The catheter length may vary from a few centimeters for peripheral access to many centimeters for central access. The catheter is commonly incorporated into a catheter adapter to aid in the ease of use, accessibility and utility of the catheter. A catheter adapter may be adapted to house one end of the catheter such that one end of the catheter is supported by the catheter adapter and the body and tip of the catheter extends beyond a first end of the catheter adapter. A catheter adapter generally further includes a second end adapted to receive additional infusion components for use with the catheter. For example, the second end of a catheter adapter may include a set of threads for attaching an intravenous line or for coupling a syringe to the catheter adapter thereby providing access to the patient's vasculature via the attached catheter.

The catheter may be inserted transcutaneously. When inserted transcutaneously, the insertion of the catheter is commonly aided by an introducer needle. The introducer needle is commonly housed inside the lumen of the catheter such that the gauge of the needle approximates the inner diameter of the catheter. The needle is positioned within the catheter such that the needle tip extends beyond the tip of the catheter whereby the needle is used to penetrate the patient's vein and provide an opening for insertion of the catheter.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal, fluid infusion, or probe access.

Blood withdrawal or infusion using the catheter may be difficult for several reasons, particularly when a dwelling time of the catheter within the patient is more than one day. For example, when the catheter is left inserted in the patient for a prolonged period of time, the catheter may be more susceptible to narrowing, collapse, kinking, blockage by debris (e.g., fibrin, platelet clots, or thrombus), and adhering of a tip of the catheter to the vasculature. Due to this, catheters may often be used for acquiring a blood sample at a time of catheter placement but are much less frequently used for acquiring a blood sample during the catheter dwell period. Therefore, when a blood sample is desired, an additional needle stick is used to provide vein access for blood collection, which may be painful for the patient and result in higher material costs.

### SUMMARY OF THE INVENTION

In one aspect or embodiment, an instrument configured to be inserted through a vascular access device includes a helical coil formed by a wire, with the helical coil defining a central passage, and a core wire extending along a longitudinal axis of the helical coil through at least a portion of the central passage of the helical coil, where at least a portion of the core wire includes a planar surface extending in a direction along the longitudinal axis and a semi-cylindrical surface extending in a direction along the longitudinal axis.

The core wire may include a first transverse cross-sectional shape, with the first transverse cross-sectional shape having a linear section and a semi-circular section. The core wire may include a second transverse cross-sectional shape, where the second transverse cross-sectional shape is circular. The first transverse cross-sectional shape may be less than 33% of a transverse cross-sectional area of the central passage. The core wire may taper in size from the second transverse cross-sectional shape to the first transverse cross-sectional shape. The semi-cylindrical surface may have a radius less than or equal to an inner radius of the helical coil. The planar surface may extend along a chord of the central passage.

In one aspect or embodiment, a vascular access system includes a catheter adapter including a catheter configured to be inserted into a patient's vasculature, an instrument advancement device coupled to the catheter adapter, where the instrument advancement device includes the instrument of any of the aspect or embodiments discussed above, and where the instrument advancement device is configured to advance the instrument from a retracted position to an advanced position beyond a distal end of the catheter. The helical coil of the instrument is configured to extend beyond the distal end of the catheter when the instrument is in the advanced position.

In one aspect or embodiment, an instrument configured to be inserted through a vascular access device includes a helical coil formed by a wire, with the helical coil defining a central passage, and a core wire extending along a longitudinal axis of the helical coil through at least a portion of the central passage of the helical coil, where the core wire includes a first wire having a first diameter and a second wire having a second diameter, with the first diameter larger than the second diameter. At least a portion of the second wire extends within the central passage. The first wire is attached to the second wire to form the core wire.

The first wire and the second wire may overlap in a direction extending along the longitudinal axis. An end of the first wire may be attached to an end of the second wire. The first core may be attached to the second wire via at least one of welding, brazing, soldering, and adhesive.

In one aspect or embodiment, an instrument configured to be inserted through a vascular access device includes a helical coil formed by a wire, with the helical coil defining a central passage, and a core wire extending along a longitudinal axis of the helical coil through at least a portion of the central passage of the helical coil, where at least a portion of a length of the core wire has a C-shaped transverse cross-section.

An outer surface of the core wire may have a radius less than or equal to an inner radius of the helical coil. The core wire may include a first portion including the length of the core wire with the C-shaped transverse cross-section and a second portion having a circular transverse cross-section.

In one aspect or embodiment, an instrument configured to be inserted through a vascular access device includes a tubular member having a first end and a second end positioned opposite the first end, with the tubular member defining a central passage and a plurality of sidewall openings in fluid communication with the central passage. The plurality of sidewall openings are positioned from the first end to the second end of the tubular member.

The instrument may include a core wire attached to the first end of the tubular member. The instrument may include a proximal member extending from and formed integrally with the tubular member, with the proximal member having a continuous, uninterrupted sidewall. The tubular member may be formed from a shape memory alloy.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1A is a perspective view of a vascular access system according to one aspect or embodiment of the present application, showing a retracted position of an instrument;

FIG. 1B is a perspective view of the vascular access system of FIG. 1, showing an advanced position of an instrument;

FIG. 1C is a partial perspective view of the vascular access system of FIG. 1, showing an advanced position of an instrument;

FIG. 1D is a partial cutaway view of the vascular access system of FIG. 1, showing an advanced position of an instrument;

FIG. 1E is a cross-sectional view of the vascular access system of FIG. 1, showing an advanced position of an instrument within a patient's vasculature;

FIG. 2 is a perspective view of an instrument of the vascular access system of FIG. 1 according to one aspect or embodiment of the present application;

FIG. 3 is a transverse cross-sectional view of the instrument of FIG. 2;

FIG. 4 is a transverse cross-sectional view of the instrument of FIG. 2;

FIG. 5 is a perspective view of an instrument according to a further aspect or embodiment of the present application;

FIG. 6 is a perspective view of an instrument according to a further aspect or embodiment of the present application;

FIG. 7 is a schematic view of an instrument according to a further aspect or embodiment of the present application;

FIG. 8 is a transverse cross-sectional view of the instrument of FIG. 7;

FIG. 9 is a partial perspective view of an instrument according to a further aspect or embodiment of the present application, showing an intermediate portion of the instrument;

FIG. 10 is a partial perspective view of the instrument of FIG. 9, showing an end of the instrument;

FIG. 11 is a side view of an instrument according to a further aspect or embodiment of the present application; and

FIG. 12 is a perspective view of the instrument of FIG. 11.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

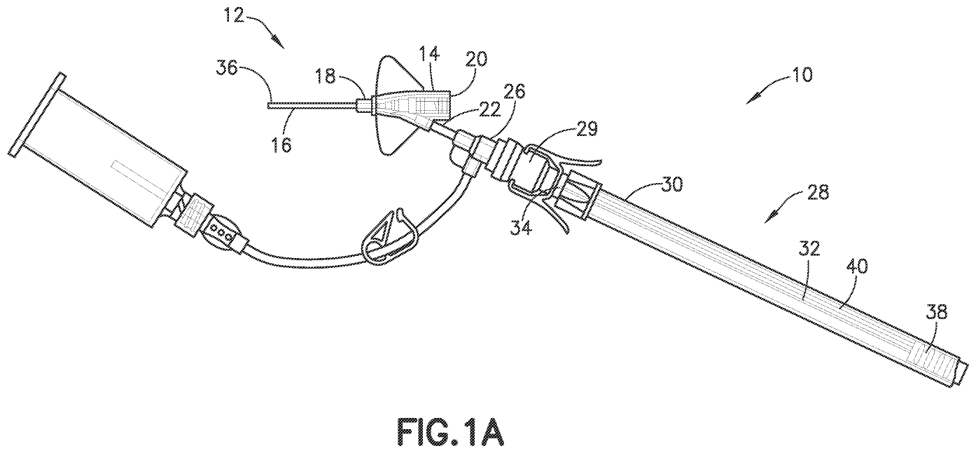

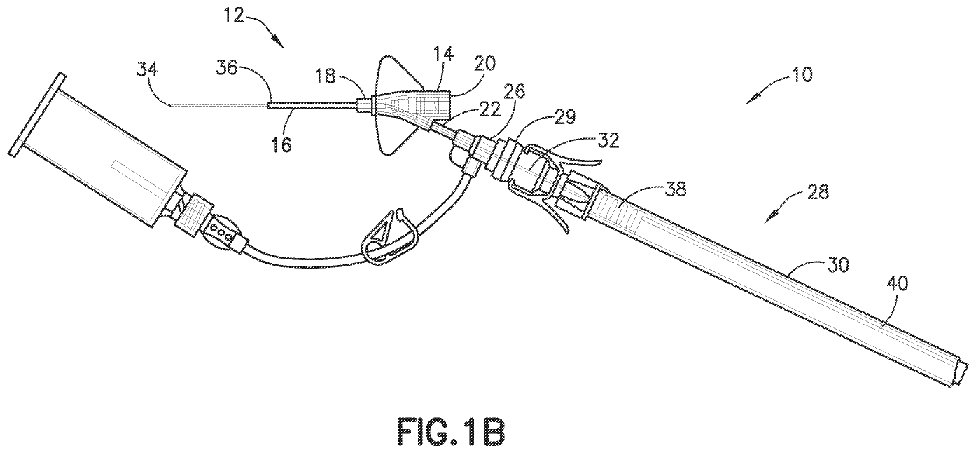

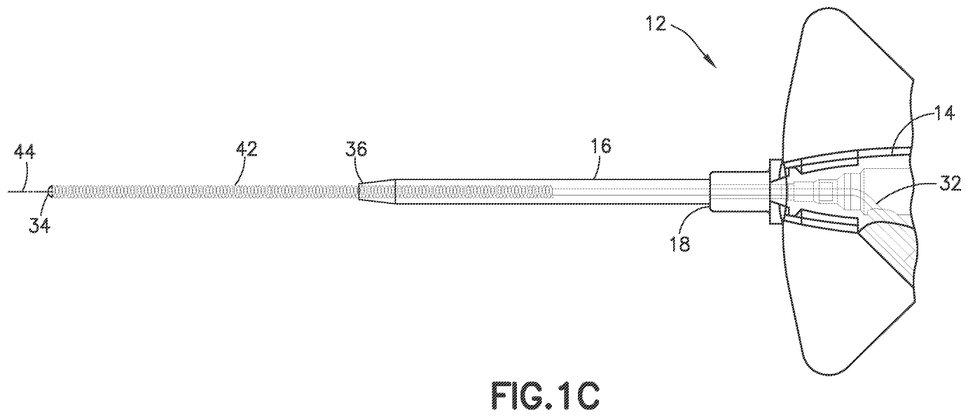

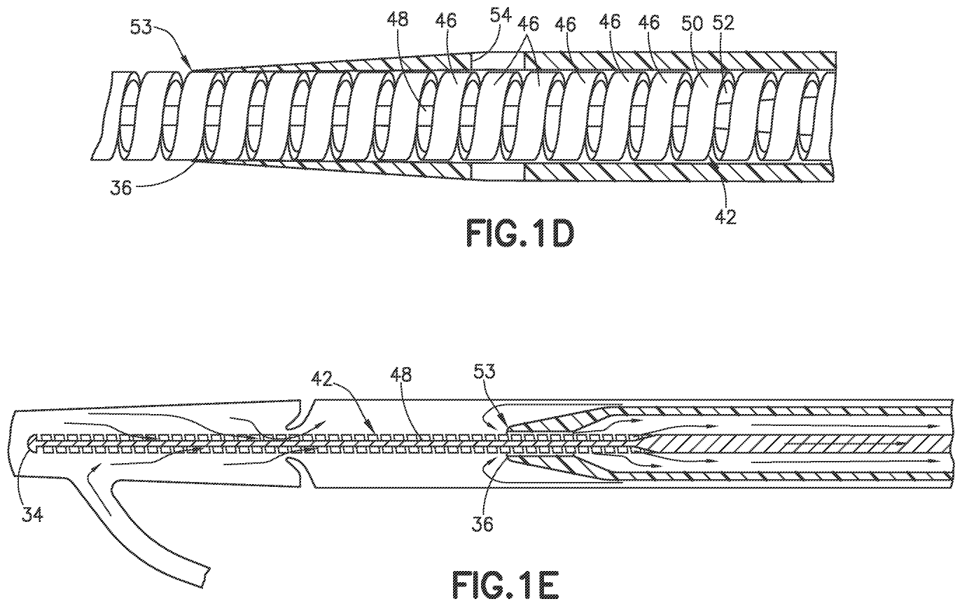

Referring to FIGS. 1A-4, in one aspect or embodiment, a vascular access system 10 includes a catheter assembly 12, which may include a catheter adapter 14 and a catheter 16. The catheter 16 may be a peripheral intravenous catheter, a peripherally-inserted central catheter, or a midline catheter. In some aspects or embodiments, the catheter adapter 14 includes a distal end 18, a proximal end 20, and a lumen extending through the distal end 18 and the proximal end 20. In some aspects or embodiments, the catheter 16 extends distally from the distal end 18 of the catheter adapter 14. The catheter adapter 14 may be integrated with an extension tube 22, which may extend from a side port 24 of the catheter adapter 14. In some aspects or embodiments, an adapter 26, such as a Y-adapter or a T-adapter, for example, may be coupled to a proximal end of the extension tube 22. An instrument advancement device 28 may be coupled to the catheter assembly 12 in various ways. In one aspect or embodiment, the instrument advancement device 28 is coupled to a port of the adapter 26. In one aspect or embodiment, the instrument advancement device 28 is coupled to a needleless connector 29 disposed between the port of the adapter 26 and the instrument advancement device 28. The instrument advancement device 28 may also be coupled to the proximal end 20 of the catheter adapter 14. In some aspects or embodiments, another extension tube and/or a blood collection device adapter may be coupled to another port of the adapter 26. The blood collection device adapter may receive a blood collection device, such as, for example, a syringe or a blood collection tube.

The instrument advancement device 28 may include a housing 30 configured to couple to the catheter assembly 12. The instrument advancement device 28 includes an instrument 32. In some aspects or embodiments, the instrument advancement device 28 may include any suitable delivery device. Some examples of instrument advancement devices that may be used with the instrument 32 are described further in in U.S. patent application Ser. No. 16/037,246, filed Jul. 17, 2018, entitled “EXTENSION HOUSING A PROBE OR INTRAVENOUS CATHETER,” U.S. patent application Ser. No. 16/388,650, filed Apr. 18, 2019, entitled “INSTRUMENT DELIVERY DEVICE HAVING A ROTARY ELEMENT,” U.S. patent application Ser. No. 16/037,319, filed Jul. 17, 2018, entitled “MULTI-DIAMETER CATHETER AND RELATED DEVICES AND METHODS,” U.S. patent application Ser. No. 16/502,541, filed Jul. 3, 2019, entitled “DELIVERY DEVICE FOR A VASCULAR ACCESS INSTRUMENT,” U.S. patent application Ser. No. 16/691,217, filed Nov. 21, 2019, entitled “SYRINGE-BASED DELIVERY DEVICE FOR A VASCULAR ACCESS INSTRUMENT,” U.S. Patent Application No. 62/794,437, filed Jan. 18, 2019, entitled “CATHETER DELIVERY DEVICE AND RELATED SYSTEMS AND METHODS,” and U.S. Patent Application No. 62/830,286, filed Apr. 5, 2019, entitled “VASCULAR ACCESS INSTRUMENT HAVING A FLUID PERMEABLE STRUCTURE AND RELATED DEVICES AND METHODS,” which are each incorporated by reference in their entirety.

In some aspects or embodiments, the instrument advancement device 28 may be configured to introduce the instrument 32 into the catheter assembly 12. In response to the instrument 32 being introduced into the catheter assembly 12, the instrument 32 may access a fluid path of the catheter assembly 12 and/or the instrument 32 may extend through the catheter assembly 12 to access the vasculature of a patient, as shown in FIG. 1E. The instrument advancement device 28 may be configured to advance the instrument 32 between a retracted position, illustrated, for example, in FIG. 1A, to an advanced position, illustrated, for example, in FIG. 1B. In some aspects or embodiments, a distal tip 34 of the instrument 32 may be disposed distal to a distal end 36 of the catheter 16 in response to the instrument 32 being in the advanced position. In some aspects or embodiments, in response to the instrument 32 being in the retracted position, the distal tip 34 of the instrument 32 may be disposed within the housing 30. A proximal end of the instrument 32 may be coupled to an advancement tab 38, which may be gripped and moved along a slot 40 by a user to move the instrument 32 between the retracted position and the advanced position. The advancement tab 38 may extend through the slot 40, and a portion of the advancement tab 38 coupled to the proximal end of the instrument 32 may be within the housing 30.

In some aspects or embodiments, the catheter 16 may be constructed of fluorinated ethylene propylene, TEFLON™, silicon, thermoplastic elastomer, thermoplastic polyurethane, a fluorinated polymer, a hydrophilic material, a hydrophobic material, an anti-fouling material, or another suitable material. In some aspects or embodiments, the catheter 16 may include an anti-thrombogenic coating. In some aspects or embodiments, all or a portion of the instrument 32 may be constructed of metal or another suitable material. In some aspects or embodiments, the distal end 36 of the catheter 16 may be symmetric or asymmetric.

Referring now to FIGS. 1C-1E, in some embodiments, the instrument 32 may include a helical coil 42 formed by a wire wound around an axis 44 into multiple loops 46. The wire may be of various cross-sectional shapes, including flat, round, oval, half-round, etc. In some aspects or embodiments, the helical coil 42 may be constructed of metal or another suitable material. In some aspects or embodiments, the helical coil 42 may be disposed at a distal end of the instrument 32 and/or adjacent the distal tip 34. In some aspects or embodiments, each of the loops 46 of the helical coil 42 may be spaced apart from a next adjacent loop of the loops 46, which may facilitate fluid permeability of the distal end of the instrument 32. The helical coil 42 may provide multiple fluid pathways along a length of the instrument 32, which may facilitate entry of blood into the catheter assembly 12 from a portion of the vasculature more distant from the catheter 16. In some aspects or embodiments, the helical coil 42 and the fluid pathways along the length of the instrument 32 may facilitate increased flow rates of fluid through the instrument 32 and the catheter 16. In some aspects or embodiments, the helical coil 42 and the fluid pathways along the length of the instrument 32 may facilitate a reduced blood collection time. In some aspects or embodiments, the helical coil 42 may reduce a shear stress and related risk of hemolysis of blood moving into and/or through the catheter 16. In some aspects or embodiments, the helical coil 42 may facilitate soft and gentle contact with a wall of the vasculature in response to insertion of the instrument 32 into the vasculature. In one aspect or embodiment, the instrument 32 includes a core wire 48 extending through at least a portion of the helical coil 42.

In some aspects or embodiments, the wire may include a first side 50 and a second side 52 opposite and parallel to the first side 50 prior to the wire being wound around the axis 44 into the loops 46 during manufacture. The first side 50 forms an outer surface of the helical coil 42. The second side 52 forms an inner surface of the helical coil 42. The core wire 48 may be coupled to the inner surface of the helical coil 42. In some aspects or embodiments, the wire may increase an inner diameter of the helical coil 42 to facilitate an increase fluid flow rate through the instrument 32. The wire may increase the inner diameter of the helical coil 42 and still allow an outer diameter of the helical coil 42 to be a same length as an outer diameter of a standard vascular access instrument.

Referring to FIG. 1D, the distal end 36 of the catheter 16 includes a distal opening 53. In some aspects or embodiments, the coil 42 may extend through the distal opening 53 of the catheter 16 in response to the instrument 32 being in the advanced position. The outer diameter of the helical coil 42 may be less than a diameter of the distal opening 53, which may create a gap through which fluid may flow. The distal end 36 of the catheter 16 may include one or more diffuser holes 54, which may be aligned with a portion of the instrument 32 that includes the coil 42 to facilitate blood flow into the catheter assembly 12 and/or fluid infusion into the vasculature.

In some aspects or embodiments, the dimensions of the helical coil 42 may vary based on a gauge-size of the catheter 16, a stiffness of the instrument 32, a spacing between each of the loops 46 of the helical coil 42, a number or size of fluid pathways along the length of the instrument 32, or another factor.

In some aspects or embodiments, the distal tip 34 may be rounded or blunt, which may prevent injury to the vasculature. The distal tip 34 may be disposed at a distal end of the core wire 48 and/or monolithically formed with the distal end of the core wire 48 as a single unit. Additionally or alternatively, in some embodiments, the distal tip 34 may be coupled to a distal end of the helical coil 42.

In some aspects or embodiments, the distal tip 34 may be rounded or blunt. The distal tip 34 may be disposed at a distal end of the elongated strip 55 and/or monolithically formed with the distal end of the elongated strip 55 as a single unit. Additionally or alternatively, in some aspects or embodiments, the distal tip 34 may be coupled to a distal end of the helical coil 42.

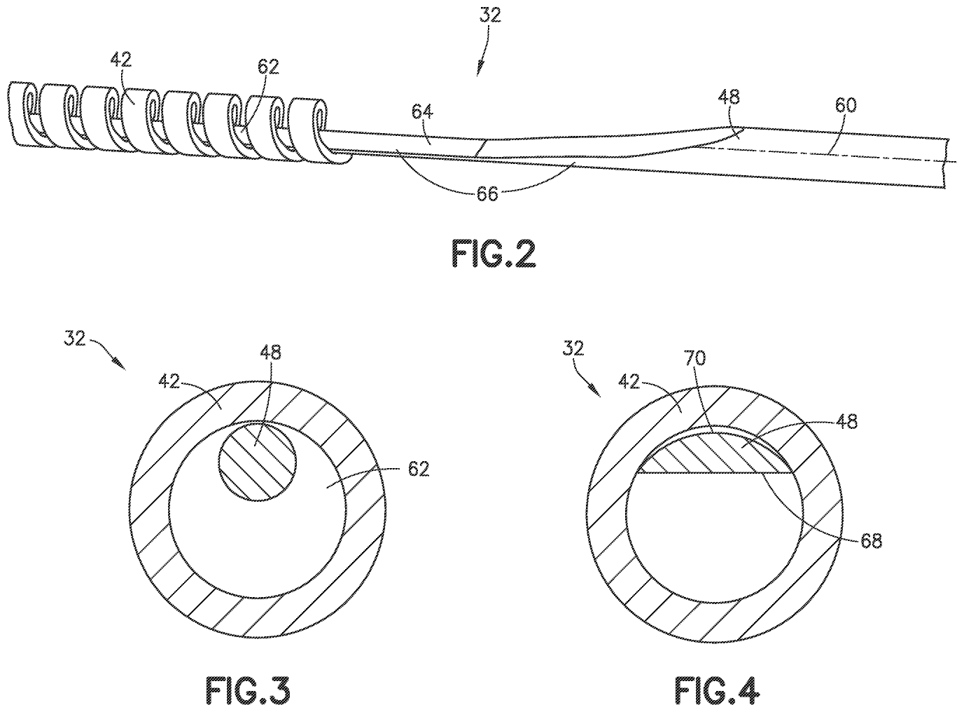

Referring to FIGS. 2-4, in one aspect or embodiment, the core wire 48 extends along a longitudinal axis 60 of the helical coil 42 through at least a portion of a central passage 62 of the helical coil 42, with at least a portion of the core wire 48 including a planar surface 64 extending in a direction along the longitudinal axis 60 and a semi-cylindrical surface 66 extending in a direction along the longitudinal axis 60. As shown in FIG. 4, the core wire 48 has a first transverse cross-sectional shape, with the first transverse cross-sectional shape having a linear section 68 and a semi-circular section 70. As shown in FIG. 3, the core wire 48 has a second transverse cross-sectional shape. The second transverse cross-sectional shape is circular. The first transverse cross-sectional shape is less than 33% of a transverse cross-sectional area of the central passage 62. As shown in FIG. 2, the core wire 48 tapers in size from the second transverse cross-sectional shape to the first transverse cross-sectional shape. In certain aspects or embodiments, the radius of the semi-cylindrical surface 66 is smaller than or equal to an inner radius of the helical coil 42 and the planer surface 64 extends along a chord of the central passage 62. In one aspect or embodiment, a length of the linear section 68 is smaller than a diameter of the central passage 62. In one aspect or embodiment, the first transverse cross-sectional shape is D-shaped. The planar surface 64 of the core wire 48 may be formed by grinding one side of a round wire or skiving a round wire. The planar surface 64 of the core wire 48 is configured to increase the flexibility of at least a portion of the instrument 32 while ensuring a sufficient portion of the central passage 62 remains open to maximize fluid flow through the central passage 62.

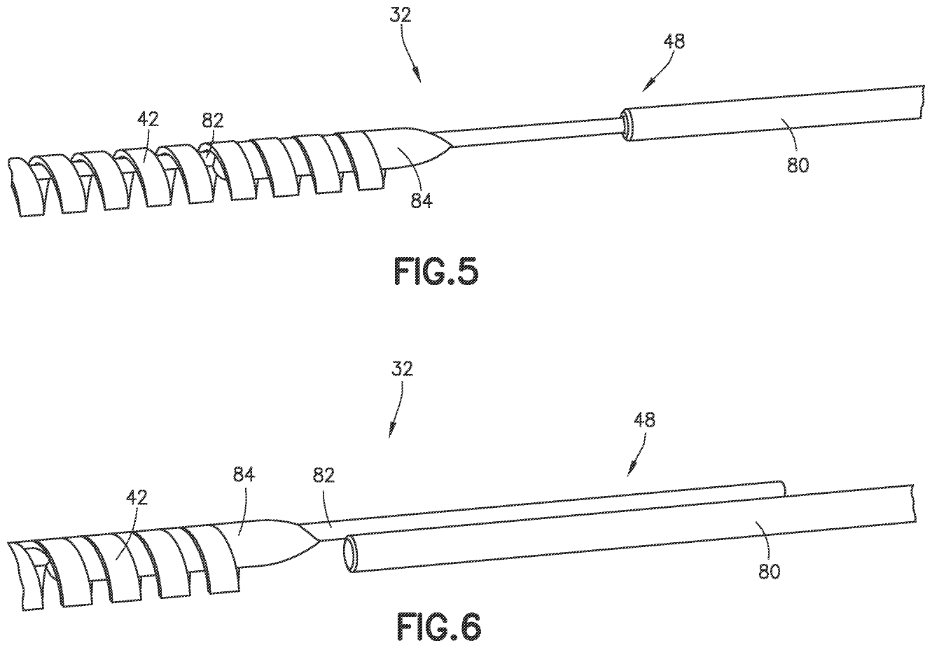

Referring to FIGS. 5 and 6, in a further aspect or embodiment, the core wire 48 includes a first wire 80 having a first diameter and a second wire 82 having a second diameter, where the first diameter is larger than the second diameter. At least a portion of the second wire 82 extends within the central passage 62, with the first wire 80 attached to the second wire 82 to form the core wire 48. As shown in FIG. 6, in one aspect or embodiment, the first wire 80 and the second wire 82 overlap in a direction extending along the longitudinal axis 60. As shown in FIG. 5, in one aspect or embodiment, an end of the first wire 80 is attached to an end of the second wire 82. The first wire 80 may be attached to the second wire 82 via at least one of welding, brazing, soldering, and adhesive. In some aspects or embodiment, the core wire 48 includes a connection 84, such as an adhesive connection, configured to hold the second wire 82 to the side of the helical coil 42. Although the connection 84 is shown as being larger in diameter than the first wire 80 and the second wire 82 for clarity, the connection 84 may be the same diameter or smaller than the diameter of the first wire 80.

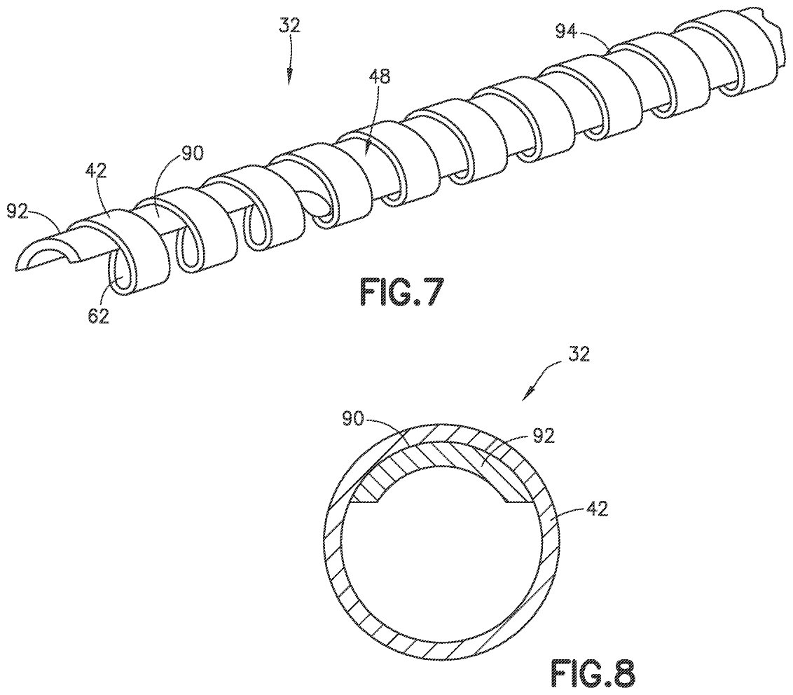

Referring to FIGS. 7 and 8, in a further aspect or embodiment, at least a portion of a length of the core wire 48 has a C-shaped transverse cross-section. An outer surface 90 of the core wire 48 may have a radius less than or equal to an inner radius of the helical coil 42. The core wire 48 may include a first portion 92 including the length of the core wire 48 with the C-shaped transverse cross-section and a second portion 94 having a circular transverse cross-section. The C-shaped transverse cross-section provides stiffness and support for insertion of the instrument while maintaining a sufficient area within the central passage 62 to maximize a flow of fluid through the central passage 62.

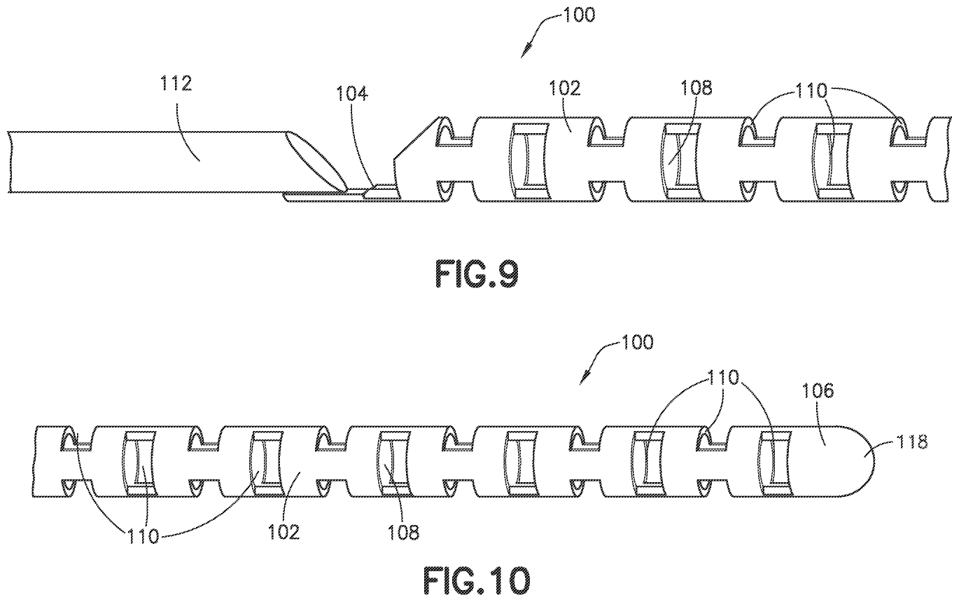

Referring to FIGS. 9-12, an instrument 100 according to further aspect or embodiments of the present application is shown. The instrument 100 may generally function in the same manner as the instrument 32 discussed above. Instead of providing the helical coil 42, the instrument 100 includes a tubular member 102 having a first end 104 and a second end 106 positioned opposite the first end 104, with the tubular member 102 defining a central passage 108 and a plurality of sidewall openings 110 in fluid communication with the central passage 108. The plurality of sidewall openings 110 are positioned from the first end 104 to the second end 106 of the tubular member 102. The fluid path of the instrument 100 of FIGS. 9-12, may be contained within the tubular member 102 rather than permitted to flow outside in the catheter lumen as shown in FIG. 1E. The sidewall openings 110 may be only distal to the catheter tip, to reduce the chance of fluid mixing and/or contamination from within the catheter, or both distal and proximal to the catheter tip. In this manner, a pathway to a blood draw device, such as the instrument advancement device 28, can be provided, such as a pathway that extends through the instrument advancement device 28 rather than extending through the adapter 26.

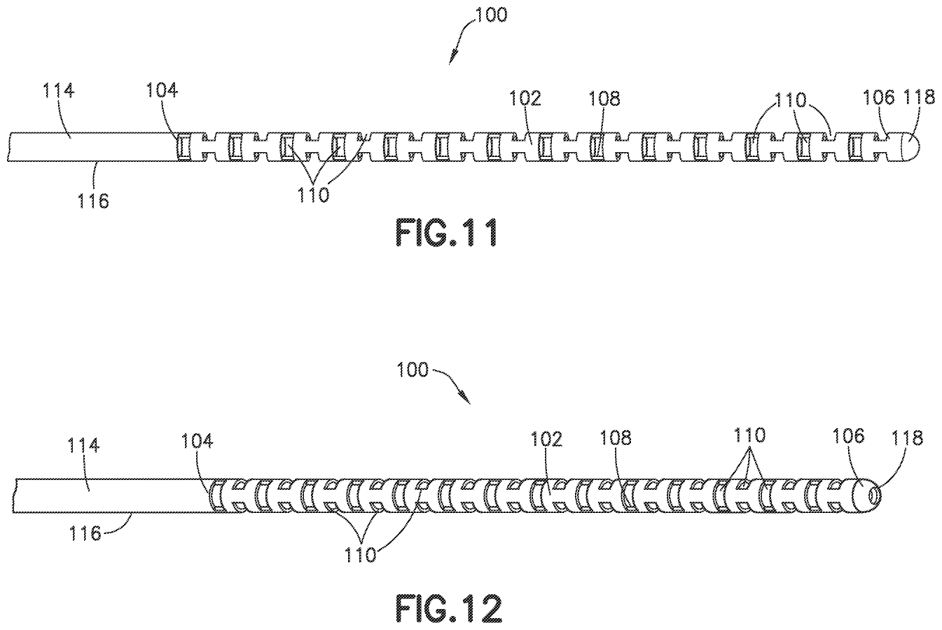

In one aspect or embodiment, as shown in FIG. 9, a core wire 112 is attached to the first end 104 of the tubular member 102. In one aspect or embodiment, as shown in FIG. 11, rather than providing the core wire 112, the instrument 100 includes a proximal member 114 extending from and formed integrally with the tubular member 102, with the proximal member 114 having a continuous, uninterrupted sidewall 116.

In one aspect or embodiment, the tubular member 102 is formed from a shape memory alloy. The plurality of sidewall openings 110 may be laser cut, or otherwise formed, into the tubular member 102. The tubular member 102 is configured to retain an outer and inner diameter without the use of a core wire positioned within the tubular member 102, which increases the size of a flow path through the central passage 108 for improved flow of fluid through the central passage 108. In one aspect or embodiment, the tubular member 102 is formed from polymeric material. The location and size of the plurality of sidewall openings 110 may be adjusted to improve manufacturability or to increase flexibility at portions of the tubular member 102, such as a distal tip 118, by increasing the size or frequency of the plurality of sidewall openings 110. In one aspect or embodiment, the shape or direct of access of the sidewall openings 110 is configured to increase flexibility at portions of the tubular member 102. For example, the sidewall openings 110 may be formed from one side or in an intermittent spiral pattern. In one aspect or embodiment, the plurality of sidewall openings 110 are configured for improved thrombus passage prevention by having smaller holes near the distal tip 118 of the instrument 100 or by providing openings 110 that are each smaller at the proximal end of each opening 110. In one aspect or embodiment, the instrument 100 is formed from metal and configured to pass an electrical signal to enable communication with a sensor (not shown). The tubular member 102 may be configured to inhibit mixing and contamination in the catheter assembly 12 and joints.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. An instrument configured to be inserted through a vascular access device, the instrument comprising:
a helical coil formed by a wire, the helical coil defining a central passage; and
a core wire extending along a longitudinal axis of the helical coil through at least a portion of the central passage of the helical coil, wherein at least a portion of the core wire comprises a planar surface extending in a direction along the longitudinal axis and a semi-cylindrical surface extending in a direction along the longitudinal axis.

2. The instrument of claim 1, wherein the core wire comprises a first transverse cross-sectional shape, the first transverse cross-sectional shape having a linear section and a semi-circular section.

3. The instrument of claim 2, wherein the core wire comprises a second transverse cross-sectional shape, and wherein the second transverse cross-sectional shape is circular.

4. The instrument of claim 2, wherein the first transverse cross-sectional shape is less than 33% of a transverse cross-sectional area of the central passage.

5. The instrument of claim 3, wherein the core wire tapers in size from the second transverse cross-sectional shape to the first transverse cross-sectional shape.

6. The instrument of claim 1, wherein the semi-cylindrical surface has a radius smaller than or equal to an inner radius of the helical coil.

7. The instrument of claim 6, wherein the planar surface extends along a chord of the central passage.

8. A vascular access system comprising:
a catheter adapter comprising a catheter configured to be inserted into a patient's vasculature;
an instrument advancement device coupled to the catheter adapter, wherein the instrument advancement device comprises a helical coil formed by a wire, the helical coil defining a central passage, and a core wire extending along a longitudinal axis of the helical coil through at least a portion of the central passage of the helical coil, wherein at least a portion of the core wire comprises a planar surface extending in a direction along the longitudinal axis and a semi-cylindrical surface extending in a direction along the longitudinal axis; and
wherein the instrument advancement device is configured to advance the instrument from a retracted position to an advanced position beyond a distal end of the catheter, the helical coil of the instrument is configured to extend beyond the distal end of the catheter when the instrument is in the advanced position.

9. An instrument configured to be inserted through a vascular access device, the instrument comprising:
a helical coil formed by a wire, the helical coil defining a central passage; and
a core wire extending along a longitudinal axis of the helical coil through at least a portion of the central passage of the helical coil, wherein the core wire comprises a first wire having a first diameter and a second wire having a second diameter, the first diameter larger than the second diameter, wherein at least a portion of the second wire extends within the central passage, and wherein the first wire is attached to the second wire to form the core wire.

10. The instrument of claim 9, wherein the first wire and the second wire overlap in a direction extending along the longitudinal axis.

11. The instrument of claim 9, wherein an end of the first wire is attached to an end of the second wire.

12. The instrument of claim 10, wherein the first wire is attached to the second wire via at least one of welding, brazing, soldering, and adhesive.

13. An instrument configured to be inserted through a vascular access device, the instrument comprising:
a helical coil formed by a wire, the helical coil defining a central passage; and
a core wire extending along a longitudinal axis of the helical coil through at least a portion of the central passage of the helical coil, wherein at least a portion of a length of the core wire has a C-shaped transverse cross-section.

14. The instrument of claim 13, wherein an outer surface of the core wire has a radius smaller or equal to an inner radius of the helical coil.

15. The instrument of claim 13, wherein the core wire comprises a first portion including the length of the core wire with the C-shaped transverse cross-section and a second portion having a circular transverse cross-section.

16. An instrument configured to be inserted through a vascular access device, the instrument comprising:
a tubular member having a first end and a second end positioned opposite the first end, the tubular member defining a central passage and a plurality of sidewall openings in fluid communication with the central passage, wherein the plurality of sidewall openings are positioned from the first end to the second end of the tubular member.

17. The instrument of claim 16, further comprising a core wire attached to the first end of the tubular member.

18. The instrument of claim 16, further comprising a proximal member extending from and formed integrally with the tubular member, the proximal member having a continuous, uninterrupted sidewall.

19. The instrument of claim 16, wherein the tubular member is formed from a shape memory alloy.
